(19) United States
(12) Patent Application Publication
(10) Pub. No.: US 2013/0090666 A1
(43) Pub. Date: Apr. 11, 2013

(54) VACUUM ASSISTED TISSUE MANIPULATION DEVICES AND SURGICAL METHODS

(71) Applicants: Christopher J. Hess, Cincinnati, OH (US); Alexander P. Kondor, Cincinnati, OH (US)

(72) Inventors: Christopher J. Hess, Cincinnati, OH (US); Alexander P. Kondor, Cincinnati, OH (US)

(73) Assignee: Ethicon Endo-Surgery, Inc., Cincinnati, OH (US)

(21) Appl. No.: 13/267,251

(22) Filed: Oct. 6, 2011

(51) Int. Cl. A61B 17/24

(52) U.S. Cl. 606/115

(57) ABSTRACT

Devices and methods for manipulating and restraining non-target tissue during a surgical procedure are disclosed. In various forms, the device includes a body member that is configurable from a collapsed position wherein the device may be inserted into the body through a small opening to an expanded position wherein the device may be used to retaining engage the non-target tissue upon the application of suction thereto. Some embodiments have at least one ferromagnetic insert therein to enable the device to be magnetically manipulated and restrained by a controlled unit located outside of the patient's body.

### FIELD OF THE INVENTION

The present application relates to methods and devices for minimally invasive therapeutic, diagnostic, or surgical procedures and, more particularly, to devices for manipulating tissue, such as, for example, soft tissue, during minimally invasive procedures.

### BACKGROUND

In a minimally invasive therapeutic, diagnostic, and surgical procedures, such as laparoscopic surgery, a surgeon may place one or more small ports into a patient's abdomen to gain access into the abdominal cavity of the patient. A surgeon may use, for example, a port for insufflating the abdominal cavity to create space, a port for introducing a laparoscope for viewing, and a number of other ports for introducing surgical instruments for operating on tissue. Other minimally invasive procedures include natural orifice transluminal endoscopic surgery (NOTES) wherein surgical instruments and viewing devices are introduced into a patient's body through, for example, the mouth, nose, or rectum. The benefits of minimally invasive procedures compared to open surgery procedures for treating certain types of wounds and diseases are now well-known to include faster recovery time and less pain for the patient, better outcomes, and lower overall costs.

During many current laparoscopic procedures it often becomes necessary to move adjacent “non-target” tissue away from the “target” tissue to facilitate manipulation and actuation of the surgical instruments on the target tissue without being hampered by non-target tissue and without injuring the non-target tissue. Magnetic anchoring and guidance systems (MAGS) have been developed for use in minimally invasive procedures to manipulate surgical instruments. MACS include an internal device attached in some manner to a surgical instrument, laparoscope or other camera or viewing device, and an external hand held device for controlling the movement of the internal device. Each of the external and internal devices has magnets which are magnetically coupled to each other across, for example, a patient's abdominal wall. While such devices may be well-suited for moving and manipulating the surgical instruments themselves, they are not designed to manipulate and/or support non-target tissue in desired orientations.

Thus, the need exists for devices, systems and methods for supporting portions of non-target tissues/organs, etc. during laparoscopic surgical procedures in unobtrusive orientations to provide better visual and physical access to the tissues/organs that are the target of the surgical procedure.

The foregoing discussion is intended only to illustrate various aspects of the related art in the field of the invention at the time, and should not be taken as a disavowal of claim scope.

### SUMMARY

In accordance with at least one general form of the present invention, there is provided a tissue manipulation device that includes a body member that has an internal space that is configured to be operably coupled to a source of suction. The body member has at least one outer surface portion that is substantially air-permeable or has at least one hole therethrough that communicates with the internal space. At least one ferromagnetic member or magnet is supported within the body member.

In accordance with another general form of the present invention, there is provided a tissue manipulation device that includes a flexible body member. In at least one form, the flexible body member comprises a first flexible side and a second flexible side that is coupled to the first flexible side to define a suction cavity therebetween. At least one first hole extends through the first flexible side into the suction cavity. At least one second hole extends through the second flexible side into the suction cavity. At least one ferromagnetic member is located on at least one of the first and second flexible sides. A flexible conduit is also in fluid communication with the suction cavity.

In accordance with other general forms of the present invention, there is provided a system for manipulating non-target tissue within the abdomen of a patient. In at least one form, the system comprises a tissue manipulation device that includes a body member that has an internal space that is configured to be operably coupled to a source of suction. The body member has at least one outer surface portion that is substantially air-permeable or has at least one hole therethrough that communicates with the internal space. At least one ferromagnetic member or at least one magnet is supported on the body member. The system further comprises a manipulatable external control unit for generating a magnetic field external to a patient's body.

In accordance with still other general forms of the present invention, there is provided a method for performing laparoscopic surgery on target tissue within a patient's abdomen. In various forms, the method includes passing a tissue manipulation device in a collapsed orientation through an opening in the patient into the patient's abdomen. The method also includes expanding the tissue manipulation device to an expanded configuration with the patient's abdomen and manipulating the expanded tissue manipulation device adjacent to non-target tissue. The method further includes applying suction to the tissue manipulation device to draw the non-target tissue into retaining engagement with at least a portion of the tissue manipulation device and moving the tissue manipulation device within the abdomen to draw the non-target tissue engaged therewith to a desired position within the abdomen.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 is a view of the front of an embodiment of the tissue manipulation device.

FIG. 2 is a side view of the tissue manipulation device of FIG. 1.

FIG. 3 is a partial cross-sectional view of a portion of a patient's abdomen with a conventional trocar device installed through the abdominal wall.

FIG. 4 is another partial cross-sectional view of the patient's abdomen of FIG. 3 with a tissue manipulation device being inserted into the abdomen through the trocar cannula.

FIG. 5 is another partial cross-sectional view of the patient's abdomen of FIGS. 3 and 4 after the tissue manipulation device has been inserted into the abdomen and has assumed its deployed configuration.

FIG. 6 is another partial cross-sectional view of the patient's abdomen of FIGS. 3-5 after the tissue manipulation device has engaged the liver and a portion of the abdominal wall.

FIG. 7 is a front view of another tissue manipulation device embodiment.

FIG. 8 is a side view of the tissue manipulation device of FIG. 7.

FIG. 9 is a front view of another tissue manipulation device embodiment.

FIG. 10 is a side view of the tissue manipulation device of FIG. 9.

FIG. 11 is a partial cross-sectional view of a portion of a patient's abdomen wherein the tissue manipulation device of FIGS. 9 and 10 is being employed to manipulate and restrain the patient's liver in a non-obtrusive position.

FIG. 12 is a front view of another tissue manipulation device embodiment.

FIG. 13 is a side view of the device of FIG. 12.

Corresponding reference characters indicate corresponding parts throughout the several views. The exemplifications set out herein illustrate various embodiments of the invention, in one form, and such exemplifications are not to be construed as limiting the scope of the invention in any manner.

### DESCRIPTION

Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

In describing and claiming the present invention, the following terminology will be used in accordance with the definitions set out below.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located farthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

As used herein, the term “biocompatible” includes any material that is compatible with the living tissues and system(s) of a patient by not being substantially toxic or injurious and not known to cause immunological rejection. “Biocompatibility” includes the tendency of a material to be biocompatible.

As used herein, the term “operatively connected” with respect to two or more components, means that operation of, movement of, or some action of one component brings about, directly or indirectly, an operation, movement or reaction in the other component or components. Components that are operatively connected may be directly connected, may be indirectly connected to each other with one or more additional components interposed between the two, or may not be connected at all, but within a position such that the operation of, movement of, or action of one component effects an operation, movement, or reaction in the other component in a causal manner.

As used herein, the term “patient” refers to any human or animal on which a suturing procedure may be performed. As used herein, the term “internal site” of a patient means a lumen, body cavity or other location in a patient's body including, without limitation, sites accessible through natural orifices or through incisions.

As used herein, the term “fluid communication” with respect to two or more components means that such components are interconnected by a lumen, pipe, conduit, passage, etc. that facilitates the passage of fluid therethrough from one component to the other component.

As used herein, the term “target tissue” refers to that tissue, organ, body part, etc. that is the primary subject of the surgical procedure. Thus, the term “non-target tissue” as used herein refers to that tissue, organ, body part, etc. located in the vicinity of the target tissue and which may obscure the target tissue or otherwise hamper performance of the surgical procedure if not moved to and restrained in a non-obscuring position.

As used herein, the term “fluid” may refer to air, liquid or other gaseous mediums.

When used as a verb herein, the term “suction” means the flow of fluid into a partial vacuum or region of lower pressure. The pressure gradient between this region and the ambient pressure will propel matter toward the low pressure area. The term “source of suction” as used herein encompasses any device capable of drawing a fluid through a conduit, lumen, pipe, passage, etc. Such devices may include, but are not limited to, vacuum pumps, suction pumps, vacuum blowers, suction systems/plenums commonly found in surgical suites, etc.

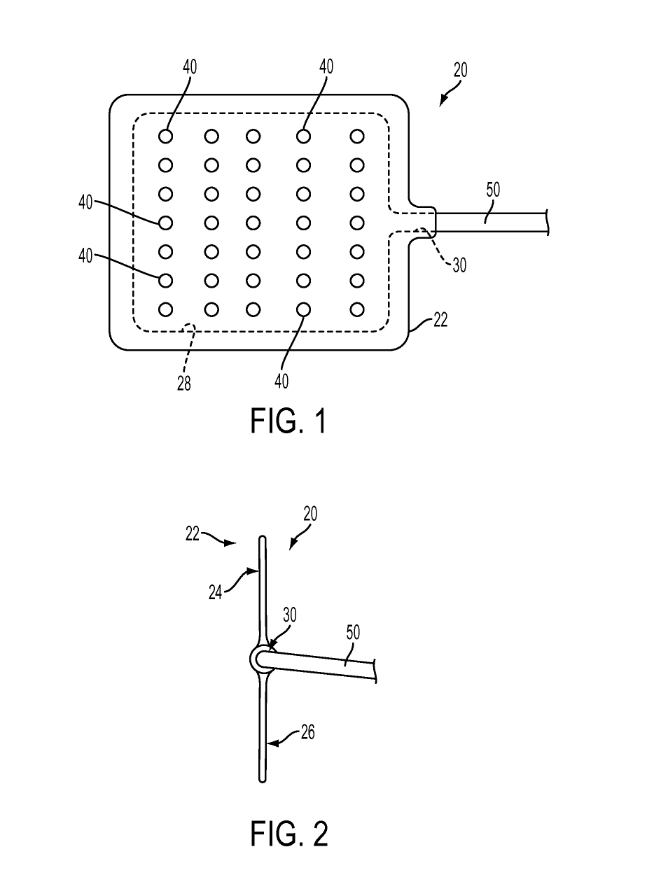

FIGS. 1 and 2 illustrate one form of a tissue manipulation device 20 of the present invention. In at least one embodiment, the tissue manipulation device 20 includes a body member 22 that is fabricated from biocompatible flexible material such as, for example, silicone rubber, flexible polymer materials, etc. The body member 22 includes a first side 24 that is attached to a second side 26 around their respective perimeters to define an internal space 28 therebetween. The body member 20 may be of one-piece molded construction or the first and second sides 24, 26 may be attached together at their respective perimeters by appropriate adhesive, welding, sealant tapes, etc. such that a substantially fluid-tight seal is established therebetween. As can also be seen in FIGS. 1 and 2, the body member 22 is configured with a suction port 30 that is in fluid communication with the internal space 28.

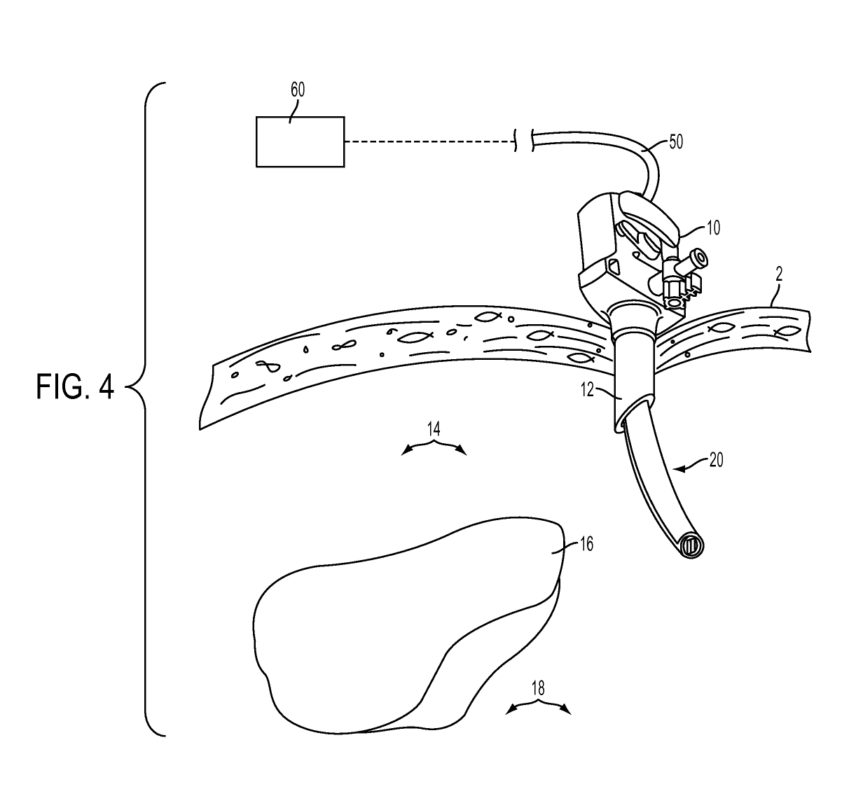

In various embodiments, the body member 22 is substantially flexible such that it may be configured in a collapsed position wherein the body member 22 may be passed through an opening or passage in the patient into an internal site or through the cannula of a trocar. For example, in at least one embodiment, the body member 22 may be rolled into a collapsed orientation (FIG. 4) that would permit the body member 22 to be inserted through the trocar cannula 12 and then opened to a “deployed” configuration. In at least one embodiment, the body member 22 is substantially planar when in the deployed configuration. In other embodiments, the body member 22 may have a shape that is complementary to a portion of the non-target tissue to be manipulated/retained when it is in the deployed configuration. In at least one embodiment, for example, the body member 22 may be fabricated from material that naturally assumes the deployed configuration after it has completely passed through the trocar cannula or other opening/passage into the internal site within the patient. In other embodiments, however, the body member 22 may be moved to the deployed configuration by means of conventional surgical instruments such as grasping devices or the like.

In at least one form, the body member 22 has at least one suction hole 40 or opening therethrough that communicates with the internal space 28. In the embodiment depicted in FIG. 1, a plurality of suction holes 40 arranged in a grid-like fashion, extend through the first side 24 such that fluid may pass from outside of the body member 22 through the suction holes 40 into the internal space 28. In one embodiment, a similar plurality of second suction holes (not shown) extend through the second side 26. The number, size, shape, and arrangement of the first and second holes (if present) may vary and are not intended to be limiting. As can be further seen in FIGS. 1 and 2, a flexible conduit 50 is attached to the suction port 30 such that it is in fluid communication with the internal space 28. In those embodiments of molded construction, for example, the holes may communicate with the suction port 30 by passages molded within the body member 22.

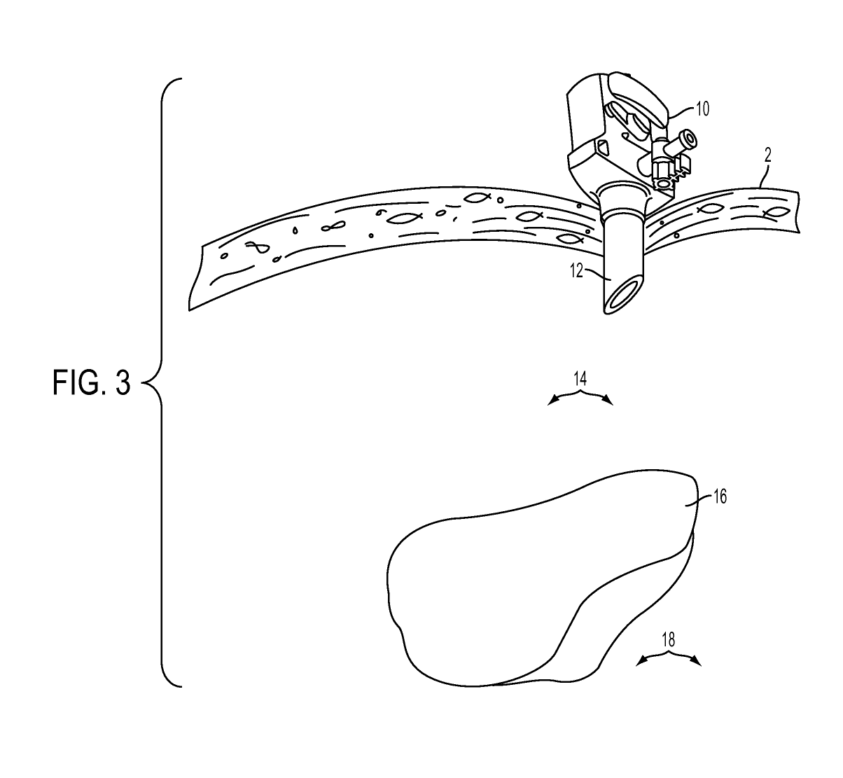

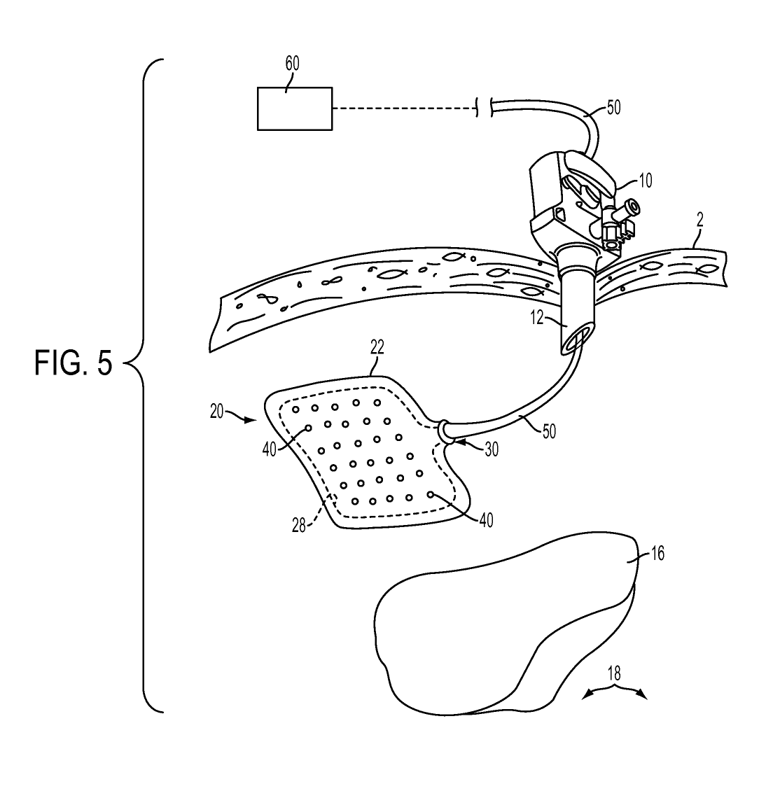

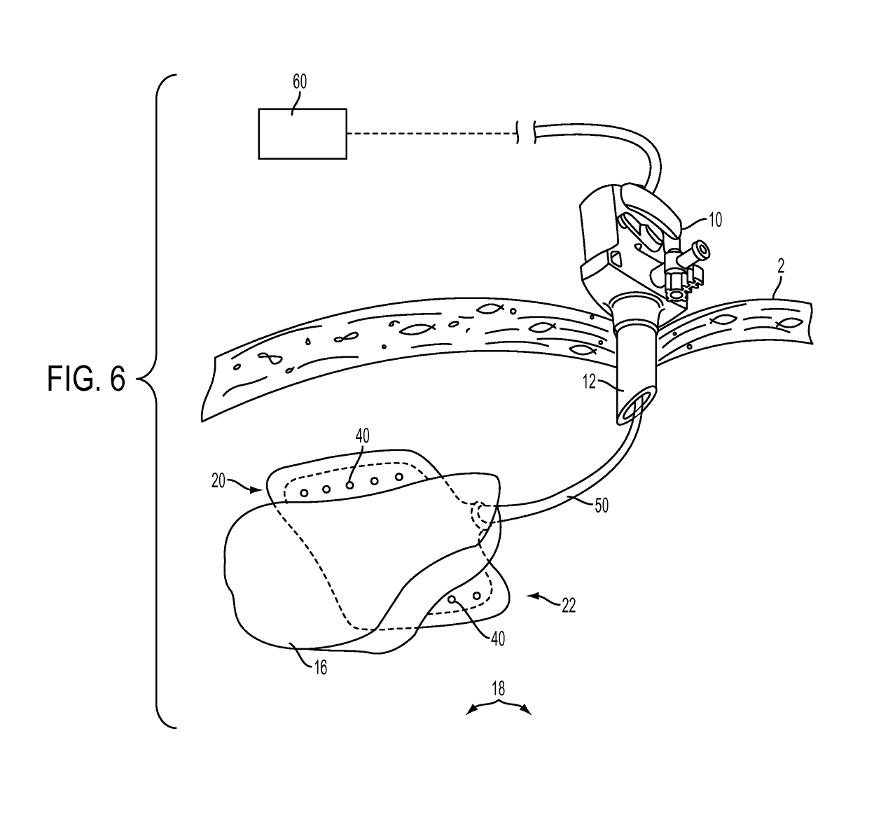

FIGS. 3-6 illustrate one method of using at least one form of the tissue manipulation device 20 of the present invention to manipulate and move a non-target organ such as the liver away from the surgical site during a laparoscopic surgical procedure. Turning first to FIG. 3, a conventional trocar 10 is installed through the patient's abdominal wall 2 such that the trocar cannula 12 protrudes therethrough into the patient's abdomen, generally designated as 14. The trocar 10 may be used to insufflate the abdomen with gas such as Co2 or the like using conventional techniques and procedures. After the abdomen has been insufflated, the tissue manipulation device 20 may be configured into a collapsed orientation and passed through the trocar cannula 12 into the abdomen 14. Prior to or after the tissue manipulation device 20 has been passed through the trocar cannula 12, the flexible conduit 50 is attached to a source of suction or vacuum 60. The source of suction 60 may comprise a vacuum line or plenum attached to a vacuum system located in the surgical suite in which the operation is being performed. However, the source of suction 60 may comprise a vacuum pump, a vacuum blower, or other source of vacuum.

In the procedure depicted in FIGS. 3-6, it is desirable to move the patient's liver 16 away from the target surgical area generally designated as 18. Once the tissue manipulation device 20 has been passed into the abdomen 14, the surgeon may use the flexible conduit 50 and/or other surgical grasping instruments passed into the abdomen through other passages or trocars (not shown) to bring the deployed tissue manipulation device 20 into close proximity or into contact with the liver 16. As used in this context, the term “in close proximity” means that the body member 22 is located sufficiently close enough to the liver such that the application of suction from the source of suction 60 through the flexible conduit 50 into the internal space 28 will draw the adjacent portion of the liver 16 into retaining engagement with the tissue manipulation device 20. Once the tissue manipulation device 20 has been moved into close proximity to or in contact with the liver 16, the surgeon may activate the source of suction 60 to permit the liver 16 to be drawn into retaining engagement with the tissue manipulation device 20. Such method may involve, for example, opening a valve in the suction line located within the surgical suite to permit suction to be applied to the flexible conduit 50. Once the tissue manipulation device 20 has been attached to the liver 16 by means of the suction passing through the holes 40, the surgeon may then manipulate the liver 16 to a position away from the surgical location 18 and adjacent to another portion of the abdominal wall 2. In embodiments wherein suction holes 40 are provided through the first and second sides 24 and 26 of the tissue manipulation device 20, the liver 16 and tissue manipulation device 20 may be manipulated into close proximity with the another portion of the abdominal wall such that the tissue manipulation device 20 is drawn into retaining engagement with the abdominal wall 2 to retain the liver in that position during surgery. Once the surgery has been completed, the surgeon may discontinue the application of suction to the tissue manipulation device 20 to permit the liver 16 to be released and moved back to its original position.

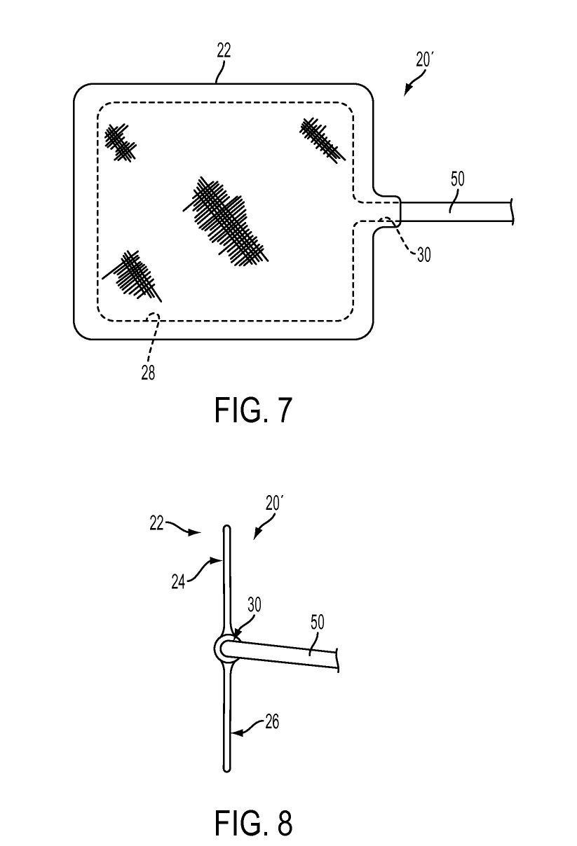

FIGS. 7 and 8 illustrate an alternative tissue manipulation device 20′ embodiment that is substantially similar to tissue manipulation device 20 described above. However, in this embodiment, at least one of the first and second side members 24 and 26 have at least one portion that is substantially air-permeable. For example, in one embodiment, both of the first and second sides 24, 26 have air permeable portions or may each be completely air-permeable. In other embodiments, only one of the first and second sides 24, 26 is air permeable. In still other embodiments, one of the first and second sides 24, 26 have at least one suction hole 40 therethrough and the other of the first and second side 24, 26 has at least one air permeable portion therein. Those embodiments having hole(s) 40 and/or air permeable portion(s) in both first and second sides 24, 26 may be used in the manner described above. Those embodiments that have hole(s) 40 and/or an air-permeable portion in only one of the first and second sides 24, 26 may be attached by suction to non-target tissue(s) and then retained in position by restraining the conduit 50 or the body member 22 in a non-obtrusive internal location by conventional means such as clamps or the like.

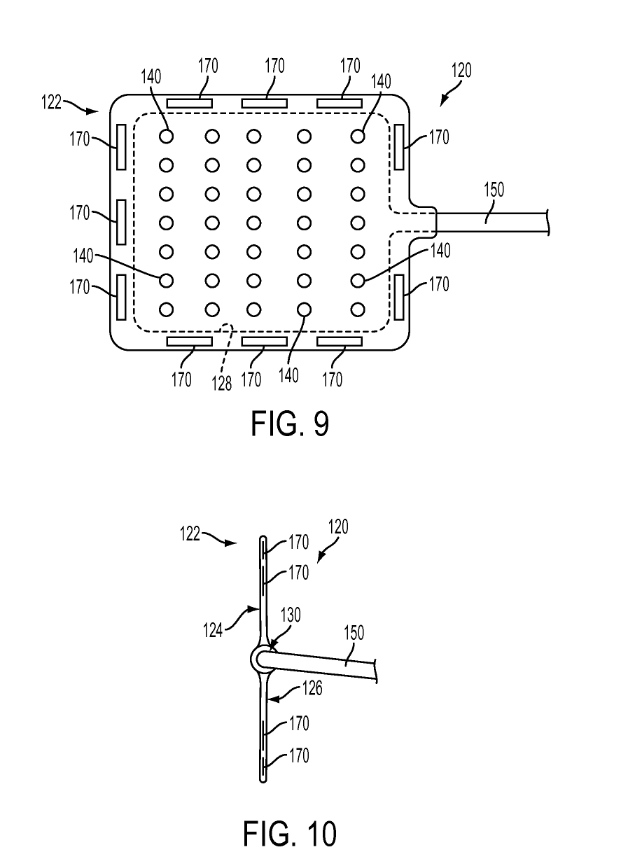

FIGS. 9 and 10 illustrate another form of a tissue manipulation device 120 of the present invention. In at least one embodiment, the tissue manipulation device 120 includes a body member 122 that is fabricated from biocompatible flexible material such as, for example, silicone rubber, flexible polymer materials, etc. The body member 122 includes a first side 124 that is attached to a second side 126 around their respective perimeters to define an internal space 128 therebetween. The body member 120 may be of one-piece molded construction or the first and second sides 124, 126 may be attached together at their respective perimeters by appropriate adhesive, welding, sealant tapes, etc. such that a substantially fluid-tight seal is established therebetween. As can also be seen in FIGS. 9 and 10, the body member 122 is configured with a suction port 130 that is in fluid communication with the internal space 128.

In various embodiments, the body member 122 is substantially flexible such that it may be configured in a collapsed position wherein the body member 122 may be passed through an opening or passage in the patient or through the cannula of a trocar. For example, in at least one embodiment, the body member 122 may be rolled into a collapsed orientation that would permit the body member 122 to be inserted through the trocar cannula and then opened to a “deployed” configuration. In at least one embodiment, the body member 122 is substantially planar when in the deployed configuration. In other embodiments, the body member 122 may have a shape that is complementary to a portion of the non-target tissue to be manipulated/retained when it is in the deployed configuration. In at least one embodiment, for example, the body member 122 may be fabricated from material that naturally assumes the deployed configuration after it has completely passed through the trocar cannula or other opening/passage in the patient. In other embodiments, however, the body member 122 may be moved to the deployed configuration by means of conventional surgical instruments such as grasping devices or the like.

In at least one form, the body member 122 has at least one suction hole 140 or opening therethrough that communicates with the internal space 128. In the embodiment depicted in FIG. 9, a plurality of suction holes 140 arranged in a grid-like fashion, extend through the first side 124 such that fluid may pass from outside of the body member 122 through the suction holes 140 into the internal space 128. In one embodiment, a similar plurality of second suction holes (not shown) extend through the second side 126. The number, size, shape, and arrangement of the first and second holes (if present) may vary and are not intended to be limiting. As can be further seen in FIGS. 9 and 10, a flexible conduit 150 is attached to the suction port 130 such that it is in fluid communication with the internal space 128.

In at least one embodiment, the tissue manipulation device 120 further includes at least one ferromagnetic insert 170. In a preferred embodiment, a plurality of inserts 170 are attached to or embedded within the body member 122 on the perimeter portion thereof around the internal space 128. In the embodiment depicted in FIG. 9, for example, eleven inserts 170 are employed. However, other shapes, sizes, numbers and configuration of inserts 170 may be used.

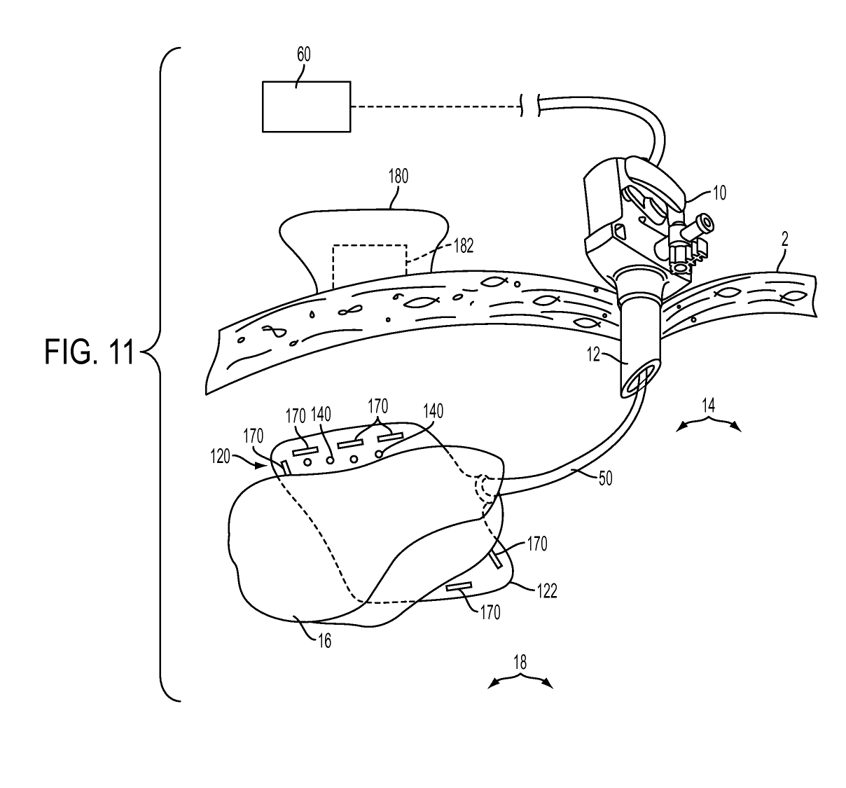

FIG. 11 illustrates one method of using at least one form of the tissue manipulation device 120 of the present invention to manipulate and move a non-target organ such as the liver 16 away from the surgical site during a laparoscopic surgical procedure. The tissue manipulation device 120 is installed into the abdomen 14 through the trocar 10 in the manner described above. Prior to or after the tissue manipulation device 120 has been passed through the trocar cannula 12 or other opening in the body, the flexible conduit 150 is attached to a source of suction or vacuum 60. The source of suction 60 may comprise a vacuum line or plenum attached to a vacuum system located in the surgical suite in which the operation is being performed. However, the source of suction 60 may comprise a vacuum pump, a vacuum blower, or other source of vacuum.

Once the tissue manipulation device 120 has been passed into the abdomen 14, the surgeon may used the flexible conduit 150 and/or other surgical grasping instruments passed into the abdomen through other passages or trocars (not shown) to bring the deployed tissue manipulation device 120 into close proximity or in contact with the liver 16. Alternatively, the tissue manipulation device 120 may be positioned using an external control unit 180 as will be discussed below. Once the tissue manipulation device 120 has been moved into close proximity with or in contact with the liver 16, the surgeon may activate the source of suction 60 to permit the liver 16 to be drawn into retaining engagement with the tissue manipulation device 120. Such action may involve, for example, opening a valve in the suction line located within the surgical suite to permit suction to be applied to the flexible conduit 150.

Once the tissue manipulation device 120 has been attached to the liver 16 by means of the suction passing through the holes 140, the surgeon may then manipulate the liver 16 to a position away from the surgical location 18 and adjacent to another portion of the abdominal wall 2 using an external source of magnetic force. In the depicted embodiment, for example, a hand-held external control unit 180 is employed. In at least one form, the external control unit 180 comprises at least one magnet, generally designated as 182, that can attract the magnetic inserts 170 in the tissue manipulation device 120. In various embodiments, the external control unit 180 may be powered through a tether that may be attached to a source of electrical power in the surgical suite. The external control unit 180 is used outside of the patient's abdomen to move and manipulate the device 120 into a non-obtrusive position and retain it there during the completion of the surgical procedure on the target tissue/organ. Once the surgery has been completed, the surgeon may discontinue the application of suction to the tissue manipulation device 120 to permit the liver 16 to be released and move back to its original position and the external control unit 180 may be moved away from the outside of the abdominal wall 2 and or the magnetic force be discontinued to enable the device 120 to be withdrawn from the patient.

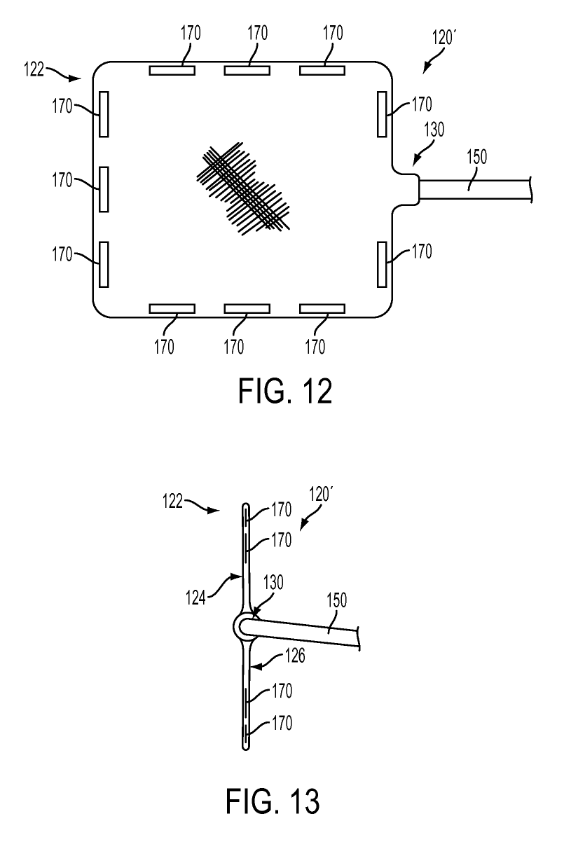

FIGS. 12 and 13 illustrate an alternative tissue manipulation device 120′ embodiment that is substantially similar to tissue manipulation device 120 described above. However, in this embodiment, at least one of the first and second side members 124 and 126 have at least one portion that is substantially air-permeable. For example, in one embodiment, both of the first and second sides 124, 126 have air permeable portions or may each be completely air-permeable.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, nose, anus, and/or vagina, for example. Minimally invasive procedures performed by the introduction of various medical devices into the patient through a natural opening of the patient are known in the art as NOTES™ procedures. Some portions of the devices may be introduced to the tissue treatment region percutaneously or through small—keyhole—incisions. In other applications, the devices may be inserted through a working channel in an endoscopic device that has been inserted into the surgical area.

The various embodiments of the present invention represent vast improvements over prior devices and methods used to manipulate and restrain non-target tissue/organs during surgical procedures. The various embodiments may be configured into a collapsed position wherein the device may be installed through a relatively small opening in the patient and then permitted to assume a more expanded or deployed configuration. Some embodiments may naturally assume the deployed configuration after they have been passed through the opening. Some embodiments may assume a configuration that is advantageously shaped with respect to the shape of the non-target tissue/organs to be manipulated and/or restrained. All materials used that are in contact with a patient are preferably made of biocompatible materials. Moreover, the various embodiments of the present invention may be operated with the services (e.g., vacuum/suction, electricity, etc.) that are generally available in conventional surgical suites. Thus, various embodiments may be effectively employed without the need of special utilities that are not normally found in a surgical suite.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A tissue manipulation device comprising:
a body member having an internal space configured to be operably coupled to a source of suction, said body member having at least one outer surface portion that is substantially air-permeable or has at least one hole therethrough that communicates with said internal space; and
at least one ferromagnetic member or at least one magnet supported within said body member.

2. The tissue manipulation device of claim 1 wherein said body member is substantially flexible.

3. The tissue manipulation device of claim 2 wherein said body member is reconfigurable between a first collapsed position and a second deployed configuration.

4. The tissue manipulation device of claim 3 wherein, when said body member is in said first collapsed position, said body member is in a rolled-up configuration and when said body member is in said deployed configuration, said body member is substantially planar.

5. The tissue manipulation device of claim 1 further comprising a conduit in fluid communication with said internal space of said body member and the source of suction.

6. The tissue manipulation device of claim 4 wherein said body is fabricated from a material such that when said body member is released from said rolled-up configuration, said body member naturally assumes said deployed configuration.

7. A tissue manipulation device comprising:
a flexible body member comprising:
a first flexible side;
a second flexible side coupled to said first flexible side to define a suction cavity therebetween;
at least one first hole through said first flexible side extending into said suction cavity; and
at least one second hole through said second flexible side extending into said suction cavity;
at least one ferromagnetic member on at least one of said first and second flexible sides and wherein said tissue manipulation device further comprises:
a flexible conduit in fluid communication with said suction cavity.

8. The tissue manipulation device of claim 7 wherein said at least one first hole comprises a plurality of first holes arranged in a first pattern in said first flexible side and wherein said at least one second hole comprises a plurality of second holes arranged in a second pattern in said second flexible side.

9. The tissue manipulation device of claim 7 wherein said body member is fabricated from a flexible material that is configurable between a first rolled-up configuration which permits said body member to be passed through an opening in a patient and a second expanded configuration.

10. The tissue manipulation device of claim 9 wherein said flexible material enables said body member to naturally assume said expanded configuration after said body member has been completely passed through the opening.

11. A system for manipulating non-target tissue within the abdomen of a patient, comprising:
a tissue manipulation device comprising:
a body member having an internal space configured to be operably coupled to a source of suction, said body member having at least one outer surface portion that is substantially air-permeable or has at least one hole therethrough that communicates with said internal space; and
at least one ferromagnetic member or at least one magnet supported on said body member, and wherein said system further comprises:
a manipulatable external control unit for generating a magnetic field external to a patient's body.

12. The system of claim 11 further comprising a flexible conduit in fluid communication with said body member and said source of suction.

13. A method for performing laparoscopic surgery on target tissue within a patient's abdomen, said method comprising:
passing a tissue manipulation device in a collapsed orientation through an opening in the patient into the patient's abdomen;
expanding the tissue manipulation device to an expanded configuration with the patient's abdomen;
manipulating the expanded tissue manipulation device adjacent to non-target tissue;
applying suction to the tissue manipulation device to draw the non-target tissue into retaining engagement with at least a portion of the tissue manipulation device; and
moving the tissue manipulation device within the abdomen to draw the non-target tissue engaged therewith to a desired position within the abdomen.

14. The method of claim 13 wherein said moving comprises:
magnetically attracting the tissue manipulation device to a magnetic manipulation unit located external to the patient's abdominal wall; and
moving the manipulation unit outside of the patient's abdomen to move the tissue manipulation device and engaged non-target tissue to the desired position within the abdomen.

15. The method of claim 13 wherein the tissue manipulation device has a suction conduit attached thereto for supplying suction from a source of suction and wherein said moving comprises manipulating the suction conduit to move the tissue manipulation device and non-target tissue in engagement therewith to the desired position.

16. The method of claim 13 further comprising temporarily retaining the tissue manipulation device in the desired position.

17. The method of claim 16 wherein said temporarily retaining comprises holding the tissue manipulation device in engagement with a corresponding portion of the abdominal wall.

18. The method of claim 17 wherein said holding comprises magnetically retaining the tissue manipulation device in the desired position with a magnetic force manipulation unit located outside of the patient's abdomen.

19. The method of claim 17 wherein said holding comprises retaining the tissue manipulation device in engagement with a corresponding portion of the abdominal wall with the suction applied to the tissue manipulation device.

20. The method of claim 13 wherein said passing comprises:
forming a passage through the patient's abdominal wall;
configuring the tissue manipulation device into a collapsed orientation; and
inserting the collapsed tissue manipulation device through the passage.
